SEQUENCE LISTING (6 sequences)

SEQ ID NO: 1 - DNA, 18 bp, Artificial Sequence
  Synthetic
gatacagccaaaaggcag

SEQ ID NO: 2 - DNA, 18 bp, Artificial Sequence
  Synthetic
atcattccatcaccagag

SEQ ID NO: 3 - DNA, 20 bp, Artificial Sequence
  Synthetic
gcaagtatgtctccctaagg

SEQ ID NO: 4 - DNA, 19 bp, Artificial Sequence
  Synthetic
aggtcacgtcttgctcctc

SEQ ID NO: 5 - DNA, 24 bp, Artificial Sequence
  Synthetic
tcgacagtcagccgcatcttcttt

SEQ ID NO: 6 - DNA, 23 bp, Artificial Sequence
  Synthetic
accaaatccgttgatccgacctt

(19) United States
(12) Patent Application Publication
(10) Pub. No.: US 2012/0190049 A1
(43) Pub. Date: Jul. 26, 2012

(54) GLYPICAN-3 TARGETING OF LIVER CANCER CELLS USING MULTIFUNCTIONAL NANOPARTICLES

(75) Inventors: Miqin Zhang, Bothell, WA (US); James O. Park, Bellevue, WA (US); Zachary Stephen, Seattle, WA (US)

(73) Assignee: University of Washington through its Center for Commercialization, Seattle, WA (US)

(21) Appl. No.: 13/316,236

(22) Filed: Dec. 9, 2011

Related U.S. Application Data
(60) Provisional application No. 61/421,954, filed on Dec. 10, 2010.

(51) Int. Cl. G01N 33/574

(52) U.S. Cl. 435/7.23

(57) ABSTRACT

Method for labeling and detecting liver cancer cells using an anti-glycipan-3 antibody as a targeting agent and a magnetic nanoparticle as an imaging agent.

### CROSS-REFERENCE TO RELATED APPLICATION

The present application claims the benefit of U.S. Provisional Patent Application No. 61/421,954, filed Dec. 10, 2010, expressly incorporated herein by reference in its entirety.

### STATEMENT REGARDING SEQUENCE LISTING

The sequence listing associated with this application is provided in text format in lieu of a paper copy and is hereby incorporated by reference into the specification. The name of the text file containing the sequence listing is 38173_SEQ_Final.txt. The text file is 1.37 KB; was created on Dec. 9, 2011, and is being submitted via EFS-Web with the filing of the specification.

### STATEMENT OF GOVERNMENT LICENSE RIGHTS

This invention was made with Government support under R01CA119408, R01CA134213, and R01EB006043 awarded by the National Institutes of Health (NIH). The Government has certain rights in the invention.

### BACKGROUND OF THE INVENTION

With over 660,000 new cases and 630,000 resultant deaths estimated in 2009, primary liver cancer, or hepatocellular carcinoma (HCC) is the fifth most prevalent malignancy and the third leading cause of cancer-related deaths worldwide. Although 80% of cases occur in regions where hepatitis B infection is endemic, the incidence of HCC in the United States is rising at epidemic proportions as a result of the rampant spread of hepatitis C in the 1950s and 1960's. HCC is a highly lethal disease as demonstrated by the equal annual incidence and mortality, and the dismal 8-month median survival without treatment is improved only by a modest 3 months when the best chemotherapy available, Sorafenib, is employed. On the contrary, when HCC is detected at an early stage, curative treatments such as surgical resection, liver transplantation, and ablative therapies can be utilized, achieving 5-year survival rates of up to 75%, highlighting the importance of early detection.

Diagnosis of early-stage HCC is heavily reliant on quality multiphase, contrast-enhanced computed tomography (CT) and magnetic resonance (MR) imaging, current gold standards. Although characteristic arterial enhancement with portal venous washout of a liver lesion on CT or MR is diagnostic for HCC, indeterminate lesions are frequently detected, prompting costly repeat imaging or biopsies with bleeding or tumor-seeding risks, all resulting in delay of diagnosis and treatment. Furthermore, eligibility for surgical resection or liver transplantation based on suboptimal scans either leads to early recurrences and poor outcomes, or missed treatment opportunities.

Several nanoparticle (NP) systems have been investigated for use in cancer diagnostics and therapeutics. HCC targeting with NPs remains challenging, as Kupffer cells, specialized macrophages dispersed throughout liver sinusoids that comprise an elaborate reticuloendothelial system (RES), take up these particles and interfere with imaging or delivery of therapeutic payloads. Much effort has been devoted to optimizing selective delivery of NPs to tumors while evading the RES, using surface modification of NPs with anti-fouling polymers such as polyethylene glycol and various tissue-specific ligands. Strategies that identify HCC-specific cell surface moieties to conjugate to have superior specificity hold the most promise. Vascular endothelial growth factor, epidermal growth factor receptor, aptamers, and small molecules such as galactose have been reported as potential targeting moieties for specific delivery of NPs and drugs to HCC cells. However, the demand for novel targeting ligands for HCC-specific delivery remains high, as no single receptor is uniformly expressed by the heterogeneous population of HCCs, and the efficiency of existing HCC-targeting ligands are less than ideal.

A need exists for diagnostic and therapeutic methods and systems that effectively target HCC-specific cell surface moieties and that enable the detection of HCC at an early stage such that patient outcomes can be improved. The present invention seeks to fulfill this need and provides further related advantages.

### SUMMARY OF THE INVENTION

The present invention provides methods and systems for imaging and detecting liver cancer cells.

In one aspect, the invention provides a method for detecting liver cancer cells. In one embodiment, the method includes:

(a) contacting liver cancer cells with a magnetic nanoparticle to provide liver cancer cells labeled with the nanoparticle, wherein the nanoparticle is associated with an anti-glycipan-3 antibody or functional fragment thereof bound to the liver cancer cells; and

(b) magnetic resonance imaging of the labeled liver cancer cells to detect the liver cancer cells, wherein the method has the capacity to detect about 1×10⁶ liver cancer cells.

In one embodiment, contacting the liver cancer cell with magnetic nanoparticle comprises administering the magnetic nanoparticle to a subject to be diagnosed or treated. In another embodiment, contacting the liver cancer cell with magnetic nanoparticle comprises administering the magnetic nanoparticle associated with an anti-glycipan-3 antibody or functional fragment thereof to a subject to be diagnosed or treated. Representative methods for administration include intravenous administration.

In one embodiment, the anti-glycipan-3 antibody is an IgG₁. Representative functional fragments of the anti-glycipan-3 antibody include Fab and F(ab′)₂ fragments.

In one embodiment, the anti-glycipan-3 antibody is covalently coupled to the nanoparticle. In another embodiment, the anti-glycipan-3 antibody is associated to the nanoparticle through an affinity binding interaction.

In one embodiment, the nanoparticle further comprises an imaging agent such as a magnetic resonance imaging agent, a fluorescence imaging agent, an ultrasound imaging agent, a radiolabel, a surface plasmon resonance imaging agent, or combinations thereof. In certain embodiments, the nanoparticle includes a core having a surface and a coating on the surface of the core, the coating comprising a copolymer comprising a chitosan and a poly(ethylene oxide) oligomer. In one embodiment, the copolymer is a graft copolymer having a chitosan backbone and poly(ethylene oxide) oligomer side chains.

In another embodiment, the invention provides a method for labeling liver cancer cells for imaging and/or detecting liver cancer. In this embodiment, the method includes:

(a) contacting liver cancer cells with an anti-glycipan-3 antibody or functional fragment thereof to provide liver cancer cells labeled with the anti-glycipan-3 antibody or functional fragment thereof, wherein the anti-glycipan-3 antibody or functional fragment thereof comprises a first binding partner;

(b) contacting the labeled liver cancer cells with a magnetic nanoparticle comprising a second binding partner to provide a nanoparticle-labeled liver cancer cells, wherein the first binding partner has a binding affinity toward the second binding partner effective to bind the nanoparticle to the liver cancer cells; and

(c) magnetic resonance imaging of the labeled liver cancer cells to detect the liver cancer cells, wherein the method has the capacity to detect about 1×10⁶ liver cancer cells.

In one embodiment, contacting the liver cancer cell with the anti-glycipan-3 antibody or functional fragment thereof includes administering the anti-glycipan-3 antibody or functional fragment thereof to a subject to be diagnosed or treated. In one embodiment, contacting the labeled liver cancer cells with a magnetic nanoparticle comprises administering the magnetic nanoparticle to a subject to be diagnosed or treated. Representative methods for administration include intravenous administration.

In certain embodiments, the first binding partner is biotin or analog or derivative thereof, and the second binding partner comprises an avidin, a streptavidin, a neutravidin, or functional fragment thereof.

In another aspect, the invention provides a monoclonal antibody effective in binding to glypican-3. In one embodiment, the anti-glycipan-3 antibody is an IgG₁. In other embodiments, the invention provides functional fragments of the anti-glycipan-3 antibody including Fab and F(ab′)₂ fragments.

In a further aspect of the invention, systems for labeling, imaging, and detecting liver cancer cells are provided. The system includes an anti-glycipan-3 antibody or functional fragment thereof associated with a magnetic nanoparticle. Functional fragments of the anti-glycipan-3 antibody include Fab and F(ab′)₂ fragments. In one embodiment, the anti-glycipan-3 antibody is covalently coupled to the nanoparticle. In another embodiment, the anti-glycipan-3 antibody is associated to the nanoparticle through an affinity binding interaction. In one embodiment, the anti-glycipan-3 antibody is an IgG₁.

### DESCRIPTION OF THE DRAWINGS

The patent or application file contains at least one drawing executed in color. Copies of this patent or patent application publication with color drawings will be provided by the Office upon request and payment of necessary fee.

The foregoing aspects and many of the attendant advantages of this invention will become more readily appreciated as the same become better understood by reference to the following detailed description, when taken in conjunction with the accompanying drawings.

FIG. 1A is a schematic illustration of the preparation of a representative nanoparticle (NP-AV-APC) useful in the methods and systems of the invention.

FIG. 1B is a schematic illustration of the preparation of a representative targeting agent (biotinylated αGPC3) useful in the methods and systems of the invention.

FIG. 1C is a schematic illustration of a representative method of the invention: two-step pre-targeting approach of cell labeling.

FIG. 2A compares the fluorescence intensity of a representative nanoparticle (NP-AV-APC) useful in the methods and systems of the invention with unlabeled nanoparticles (NP) and avidin-APC conjugate (AV-APC).

FIG. 2B illustrates the zeta potential (pH 7.0) of a representative nanoparticle (NP-AV-APC) useful in the methods and systems of the invention.

FIG. 2C illustrates the hydrodynamic size (phosphate buffered saline) of a representative nanoparticle (NP-AV-APC) useful in the methods and systems of the invention.

FIG. 2D is a transmission electron microscopy (TEM) image of oleic acid-coated nanoparticles (scale bar=20 nm).

FIG. 3 compares relative GPC3 expression in Hep G2 and HLF cells determined by real-time PCR for two non-sequence-overlapping PCR products measuring 250 (GPC3-250) and 430 (GPC3-430) base-pairs.

FIG. 4 compares normalized geometric mean fluorescence of Hep G2 and HLF cells treated with a representative nanoparticle (NP-AV-APC/αGPC3) useful in the methods and systems of the invention, APC-labeled αGPC3 (αGPC3-APC), and nanoparticles (NP-AV-APC), as determined by flow cytometry.

FIGS. 5A and 5B compare confocal fluorescence imaging of GPC3 targeting for Hep G2 cells (5A) and HLF cells (5B) untreated or treated with a representative nanoparticle (NP-AV-APC/αGPC3) useful in the methods and systems of the invention, APC-labeled αGPC3 (αGPC3-APC), and nanoparticles (NP-AV-APC).

FIGS. 6A and 6B compare in vitro magnetic resonance (MR) imaging of GPC3 targeted nanoparticle uptake: R₂ color maps (6A) and quantitative R₂ values (6B) for Hep G2 and HLF cells untreated or treated a representative nanoparticle (NP-AV-APC/αGPC3) useful in the methods and systems of the invention, APC-labeled αGPC3 (αGPC3-APC), and nanoparticles (NP-AV-APC).

FIGS. 7A-7D present flow cytometric data demonstrating GPC3 recognition by GPC3 IgG₁. FACS demonstrates significant geometric mean fluorescence shift in GPC3 IgG₁-FITC (black) (isotype control, red) bound HEPG2 (7A)/HEP3B (7B), while no shift was seen in HLF (7C)/SKHEP1 (7D).

FIGS. 8A and 8B are schematic illustrations for conjugating a representative GPC3 IgG fragment to a representative magnetic nanoparticle. MEA cleaves GPC3 IgG at the hinge disulfide bond leaving a sulfhydryl group (8A). SIA was used to conjugate the cleaved GPC3 IgG to the nanoparticle and the NHS ester of fluorophore Alexa Fluor 647 was to functionalize the amine groups on the PEG coating of Fe₃O₄ NPs.

### DETAILED DESCRIPTION OF THE INVENTION

The present invention provides methods and systems for imaging and detecting liver cancer cells. In the methods and systems, liver cancer cells are targeted by an anti-glypican-3 antibody and imaged and detected by a magnetic nanoparticle that is associated with the antibody.

In the methods and systems of the invention, glypican-3 (GPC3) is the molecular target for primary liver cancer (or hepatocellular carcinoma, HCC). GPC3 is a heparan sulfate proteoglycan essential in regulating embryonal cell growth, as evidenced by its mutation causing the Simpson-Golabi-Behmel overgrowth syndrome. While its expression is absent in normal adult tissues, GPC3 is significantly over-expressed in up to 80% of human HCC's. Attached to the cell membrane via a glycosyl-phosphatidylinositol anchor, GPC3 is readily accessible for antibody-mediated targeting and binding. Moreover, GPC3 has been shown to promote HCC growth by stimulating the canonical Wnt signaling pathway, exhibiting potential as an important therapeutic target. These attributes make GPC3 an ideal biomarker for HCC targeting.

The GPC3 ligand, a monoclonal antibody (mAb) that specifically and avidly binds native GPC3 expressed on the HCC cell surface, is the GPC3 targeting agent for the multifunctional NPs. A mouse anti-human GPC3 Immunoglobulin (IgG) with high GPC3-specific affinity was developed using a hybridoma clonal selection process. The antigen binding fragments Fab and F(ab′)₂ of the IgG are reported to exhibit smaller hydrodynamic sizes and less negative zeta potentials compared to intact IgG. The resultant decrease in steric hindrance can increase tissue penetration and tumor cell affinity. Furthermore, removal of the IgG Fc region can eliminate Fc-associated effector functions (e.g., complement fixation and non-specific cellular Fc binding). In addition to the IgG, Fab and F(ab′)₂ are also useful in the methods and systems of the invention.

In one aspect, the invention provides methods for imaging and detecting liver cancer cells. The method is highly sensitive and allows for the early detection of liver cancer. The method is significantly more sensitive than the current standards: multiphase, contrast-enhanced computed tomography (CT) and magnetic resonance (MR) imaging. Because of the increased sensitivity compared to existing diagnostic methods, detection of exceedingly small numbers of cancer cells is possible. As a result of early detection, enhanced patient outcomes are anticipated.

In one embodiment, the method includes the following steps:

(a) contacting liver cancer cells with a magnetic nanoparticle to provide liver cancer cells labeled with the nanoparticle, wherein the nanoparticle is associated with an anti-glycipan-3 antibody or functional fragment thereof bound to the liver cancer cells; and

(b) magnetic resonance imaging of the labeled liver cancer cells to detect the liver cancer cells, wherein the method has the capacity to detect about 1×10⁶ liver cancer cells.

As noted above, the method of the invention is highly sensitive and has the capacity to detect about 1×10⁶ liver cancer cells. Primary liver cancer lesions develop from a point of origin within the liver. During cancerous proliferation, the lesion grows substantially outwardly (e.g., spherically) from the point of origin. The lesion is a mass of cells, substantially all of which are liver cancer cells. Current state-of-the-art diagnostic technology (multiphase, contrast enhanced computed tomography and magnetic resonance imaging) is able to detect a liver cancer lesion once the cancer has progressed to where about 1×10⁹ liver cancer cells have amassed, typically in a volume of about one cubic centimeter (cm³). The method of the present invention provides a method for detecting liver cancer that is about 1,000 times more sensitive than the state-of-the-art technology.

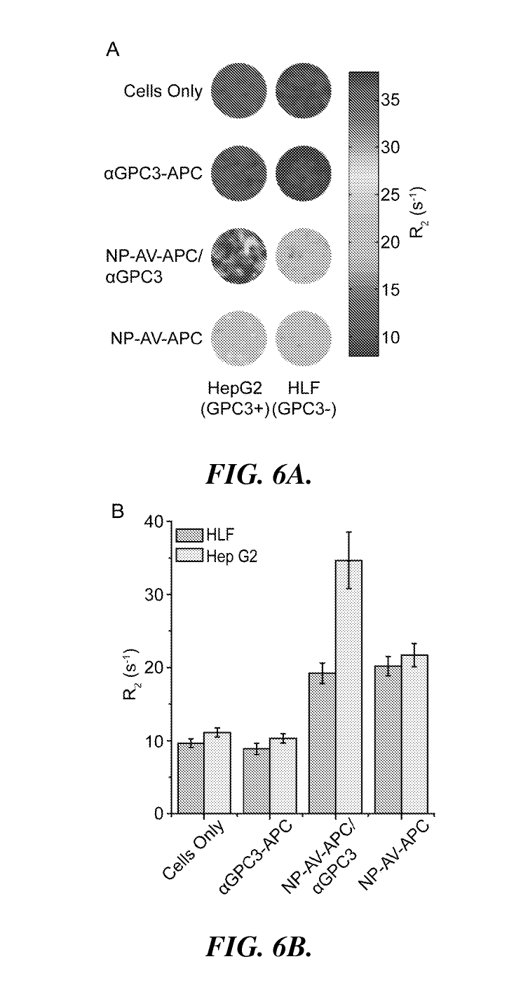

Magnetic resonance imaging of liver cancer cells is described in detail below. A representative method for magnetic resonance imaging of about 1×10⁶ liver cancer cells is described in Example 1 and the results are illustrated in FIGS. 6A and 6B.

In one embodiment of the method, contacting the liver cancer cell with magnetic nanoparticle includes administering the magnetic nanoparticles to a subject to be diagnosed or treated. The magnetic nanoparticles may be administered by intravenous administration.

In one embodiment, the magnetic nanoparticle is associated with an anti-glycipan-3 antibody or functional fragment thereof. In this embodiment, the magnetic nanoparticle and associated anti-glycipan-3 antibody or functional fragment thereof are administered together by, for example, intravenous administration. Alternatively, when the magnetic nanoparticle is not associated with the anti-glycipan-3 antibody or functional fragment thereof prior to administration, each is administered separately (e.g., intravenous administration). The nanoparticle and/or glypican-3 antibody can be administered as a composition that includes a carrier suitable for administration to a human subject. Suitable carriers include those suitable for intravenous injection (e.g., saline or dextrose).

In one embodiment, the anti-glycipan-3 antibody is associated to the nanoparticle through an affinity binding interaction (e.g., avidin-biotin). In another embodiment, the anti-glycipan-3 antibody is covalently coupled to the nanoparticle.

The magnetic nanoparticle is targeted to the liver cancer cell by the anti-glycipan-3 antibody or functional fragments thereof. Suitable functional fragments include Fab and F(ab′)₂ fragments. In one embodiment, the anti-glycipan-3 antibody is an IgG₁. A representative method for making an anti-glycipan-3 antibody is described in Example 3. Methods for making Fab and F(ab′)₂ fragments are also described in Example 3.

The nanoparticle useful in the methods and systems of the invention is a magnetic nanoparticle. As used herein, the term “magnetic nanoparticle” refers to a nanoparticle having magnetic resonance imaging activity. In certain embodiments, the nanoparticle includes a core material. For certain imaging applications, the core material is a material having magnetic resonance imaging activity (e.g., the material is paramagnetic). In certain embodiments, the core material is a magnetic material. In other embodiments, the core material is a semiconductor material. Representative core materials include ferrous oxide, ferric oxide, silicon oxide, polycrystalline silicon oxide, silicon nitride, aluminum oxide, germanium oxide, zinc selenide, tin dioxide, titanium, titanium dioxide, nickel titanium, indium tin oxide, gadolinium oxide, stainless steel, gold, and mixtures thereof.

The particle useful in the method of invention has nanoscale dimensions. Suitable particles have a physical size less than about 50 nm. In certain embodiments, the nanoparticles have a physical size from about 2 to about 30 nm. As used herein, the term “physical size” refers to the overall diameter of the nanoparticle, including core (as determined by TEM) and coating thickness. Suitable particles have a mean core size of from about 2 to about 25 nm. In certain embodiments, the nanoparticles have a mean core size of about 7 nm. As used herein, the term “mean core size” refers to the core size determined by TEM. Suitable particles have a hydrodynamic size less than about 250 nm. In certain embodiments, the nanoparticles have a hydrodynamic size from about 10 to about 250 nm. In certain embodiments, the nanoparticles have a hydrodynamic size of about 33 nm. As used herein, the term “hydrodynamic size” refers to the radius of a hard sphere that diffuses at the same rate as the particle under examination as measured by DLS. The hydrodynamic radius is calculated using the particle diffusion coefficient and the Stokes-Einstein equation given below, where k is the Boltzmann constant, T is the temperature, and η is the dispersant viscosity:

$${R}_{H} = \frac{kT}{6 π η D} .$$

A single exponential or Cumulant fit of the correlation curve is the fitting procedure recommended by the International Standards Organization (ISO). The hydrodynamic size extracted using this method is an intensity weighted average called the Z average.

In certain embodiments, the nanoparticle includes one or more imaging agents. Suitable imaging agents include magnetic resonance imaging agents, fluorescent agents, ultrasound imaging agents, radiolabels, surface plasmon resonance imaging agents. In certain embodiments, the nanoparticle has a core that renders the nanoparticle itself suitable as a magnetic resonance imaging agent, ultrasound imaging agent, radiolabel, or surface plasmon resonance imaging agent.

Suitable magnetic resonance imaging agents include iron- (e.g., iron oxide) and gadolinium-based agents.

Suitable fluorescent agents include fluorescent agents that emit visible and near-infrared light (e.g., fluorescein and cyanine derivatives). Representative fluorescent agents include fluorescein, OREGON GREEN 488, ALEXA FLUOR 555, ALEXA FLUOR 647, ALEXA FLUOR 680, Cy5, Cy5.5, and Cy7.

Suitable ultrasound imaging agents include carbon- and metal-based agents.

Suitable radiolabels includes ¹³¹I for radioimaging and ⁶⁴Cu, ¹⁸F, and ¹¹C for positron emission tomography (PET).

Suitable surface plasmon resonance imaging agents include gold-based agents.

In one embodiment, the nanoparticle includes a core having a surface with a copolymer forming a coating on the surface. The copolymer is anchored to the core surface (e.g., oxide surface) by interactions between the core surface and the amine and hydroxyl groups on the copolymer's chitosan backbone. It is believed that the coating is a multi-layered mesh that encapsulates the core.

The coating of the nanoparticle is formed from a copolymer comprising a chitosan and a poly(ethylene oxide) oligomer. In one embodiment, the copolymer is a graft copolymer having a chitosan backbone and pendant poly(ethylene oxide) oligomer side chains.

Chitosan is a linear polysaccharide composed of randomly distributed β-(1-4)-linked D-glucosamine (deacetylated unit) and N-acetyl-D-glucosamine (acetylated unit). Suitable chitosans useful in making the copolymers useful in the invention have a molecular weight (weight average, Mw) of from about 0.3 to about 50 kDa. In certain embodiments, the chitosan has a molecular weight of from about 0.5 to about 15 kDa. In one embodiment, the chitosan has a molecular weight of about 10 kDa. Suitable chitosans include oxidatively degraded chitosans prepared from commercially available chitosan.

The copolymer also includes a plurality of poly(ethylene oxide) oligomers. In one embodiment, poly(ethylene oxide) oligomers are grafted to the chitosan's backbone to provide a copolymer having pendant poly(ethylene oxide) oligomer side chains.

Suitable poly(ethylene oxide) oligomers include poly(ethylene oxides) (PEO or PEG) and poly(ethylene oxide) copolymers such as block copolymers that include poly(ethylene oxide) and poly(propylene oxide) (e.g., PEO-PPO and PEO-PPO-PEO). In one embodiment, the poly(ethylene oxide) oligomer is a poly(ethylene oxide). In certain embodiments, poly(ethylene oxide) oligomer has a molecular weight (weight average, Mw) of from about 0.3 to about 40 kDa. In others embodiments, the poly(ethylene oxide) oligomer has a molecular weight of from about 1.0 to about 10 kDa. In certain embodiments, the poly(ethylene oxide) oligomer has a molecular weight of about 2 kDa.

Representative chitosan-poly(ethylene oxide) oligomer copolymers include from about 2 to about 50 weight percent poly(ethylene oxide) oligomer. In one embodiment, the copolymer includes from about 5 to about 25 weight percent poly(ethylene oxide) oligomer.

Representative chitosan-poly(ethylene oxide) oligomer graft copolymers have a degree of poly(ethylene oxide) oligomer substitution of from about 0.01 to about 0.5. In certain embodiments, the graft copolymers have a degree of poly(ethylene oxide) oligomer substitution from about 0.01 to about 0.2. As used herein, the term “degree of substitution” or “DS” refers to the fraction of glucosamine repeating units in the chitosan that are substituted with a poly(ethylene oxide) oligomer. For DS=1.0, 100% of the glucosamine units are substituted with the poly(ethylene oxide) oligomer.

In this embodiment, the nanoparticle can be prepared by coating the core with the copolymer. In a representative method, the nanoparticle is formed by co-precipitation of iron oxide and the copolymer.

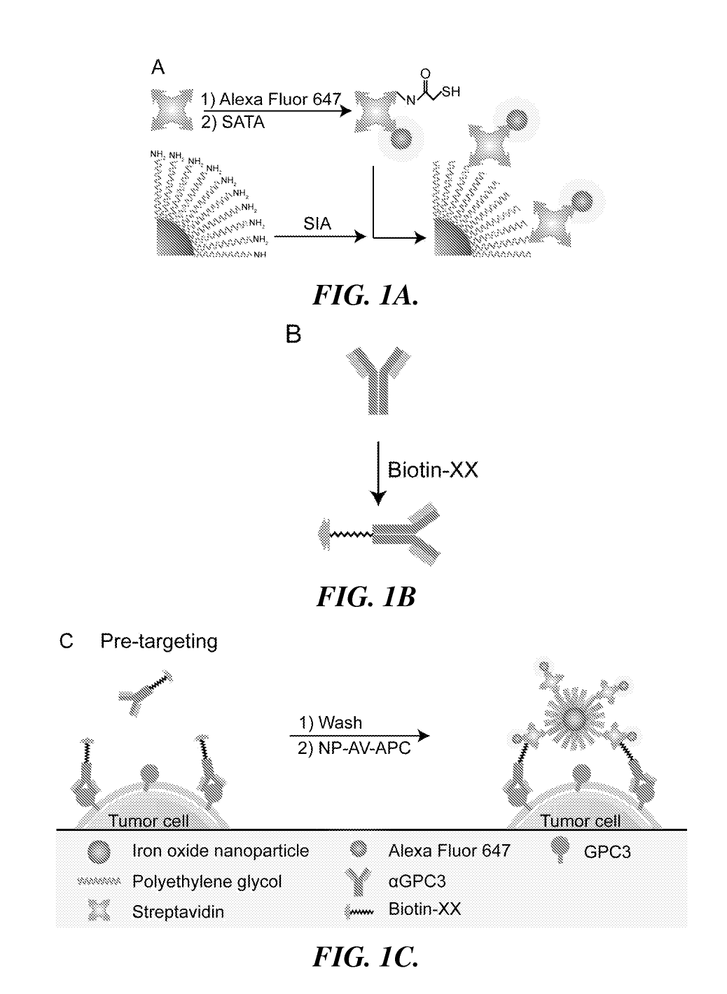

A representative magnetic nanoparticle (NP) useful in the systems and methods of the invention is a PEGylated iron oxide (Fe₃O₄) NP. PEGylated iron oxide (Fe₃O₄) NPs are from oleic acid-coated NPs generated by thermal decomposition of iron oleate complex reacted with triethoxy silylpropyl succinic anhydride (SAS) by ligand-exchange/condensation. Amine-functionalized PEG is attached to the NP via N,N-dicyclohexyl-carbodiimide (DCC)-mediated coupling. N-Succinimidyl iodoacetate (SIA) in dimethylsulfoxide (DMSO) is added to form iodoacetate functionalized NPs. The preparation of these nanoparticles is described in Example 1 and their functionalization illustrated in FIG. 1A. Their use in a representative method of the invention is illustrated schematically in FIG. 1C.

Another representative magnetic nanoparticle (NP) useful in the systems and methods of the invention is a chitosan-grafted PEG NP. For the chitosan-grafted PEG NP synthesis, Fe₃O₄ NPs are co-precipitated with a chitosan-grafted PEG polymer. The preparation of these nanoparticles is described in Example 2.

The zeta potential and hydrodynamic sizes of these particles are optimized for in vivo targeting experiments (i.e. minimizing non-specific binding and minimizing renal clearance or uptake by the reticuloendothelial system). Transmission electron microscopy demonstrated NPs of uniform size without agglomeration.

In the methods of the inventon, the liver cancer cell can be labeled with the magnetic nanoparticle directly (i.e., through the use of a magnetic nanoparticle-glypican-3 anitbody conjugate) on by a two-step method in which the cancer cell is first labeled with the antibody to provide an antibody-labeled cell that is further labeled with the nanoparticle.

In another embodmeint, the invention provides a method for labeling liver cancer cells that includes the following steps:

(a) contacting liver cancer cells with an anti-glycipan-3 antibody or functional fragment thereof to provide liver cancer cells labeled with the anti-glycipan-3 antibody or functional fragment thereof, wherein the anti-glycipan-3 antibody or functional fragment thereof comprises a first binding partner;

(b) contacting the labeled liver cancer cells with a magnetic nanoparticle comprising a second binding partner to provide a nanoparticle-labeled liver cancer cells, wherein the first binding partner has a binding affinity toward the second binding partner effective to bind the nanoparticle to the liver cancer cells; and

(c) magnetic resonance imaging of the labeled liver cancer cells to detect the liver cancer cells, wherein the method has the capacity to detect about 1×10⁶ liver cancer cells.

The magnetic nanoparticles and anti-glycipan-3 antibody or functional fragment thereof useful in this embodiment include those noted above, modified to include first and second binding partners, respectively, as described below.

In one embodiment, contacting the liver cancer cell with the anti-glycipan-3 antibody or functional fragment thereof comprises administering (e.g., intravenous administration) the anti-glycipan-3 antibody or functional fragment thereof to a subject to be diagnosed or treated. Similarly, in one embodiment, contacting the labeled liver cancer cells with a magnetic nanoparticle comprises administering (e.g., intravenous administration) the magnetic nanoparticle to a subject to be diagnosed or treated.

In accordance with this embodiment of the method, once the liver cancer cell is pre-targeted by the glypican-3 antibody of functional fragment thereof, the cancer cell is then targeted and labeled by a nanoparticle that includes a binding partner having an affinity to the pre-targeted antibody or fragment. Labeling of the pre-targeted antibody or fragment by the nanoparticle is through the affinity of the first binding partner of the antibody or fragment to the second binding partner of the nanoparticle.

Thus, in one aspect, the invention provides a method for labeling a liver cancer cell that includes contacting an antibody or fragment-labeled liver cancer cell having an affinity to a binding partner with a nanoparticle comprising the binding partner to provide a nanoparticle-labeled liver cancer cell.

The antibody or functional fragment thereof useful in this embodiment of the method includes a binding region having an affinity toward a binding partner. In the method of the invention, the nanoparticle includes the binding partner. The antibody or fragment's first binding partner has an affinity toward the nanoparticle's second binding partner, which make up a binding pair.

The antibody or fragment's first binding partner include proteinaceous regions that bind mono-, di-, and polysaccharides (e.g., carbohydrates), nucleic acids and their fragments (e.g., DNA and RNA aptamers), and small molecules (e.g., haptens). Other suitable proteinaceous binding regions include antibody binding protein A, chitin binding protein, maltose binding protein, and histidine-Ni or —Co. First binding partners also include mono-, di-, and polysaccharides (e.g., carbohydrates), nucleic acids and their fragments (e.g., DNA and RNA aptamers), and small molecules (e.g., haptens). Representative first binding partners include avidin, streptavidin, neutravidin, or functional fragments thereof. For these embodiments, the nanoparticle's binding partner is a biotin or analog or derivative thereof (e.g., desthiobiotin). Conversely, representative first binding partners include a biotin or analog or derivative thereof (e.g., desthiobiotin). For these embodiments, the nanoparticle's binding partner is an avidin, streptavidin, neutravidin, or functional fragments thereof.

In one embodiment, the first binding partner is biotin or analog or derivative thereof, and the second binding partner comprises an avidin, a streptavidin, a neutravidin, or functional fragment thereof.

The following is a description of representative nanoparticles and their use in the methods and systems of the invention.

NP Development and Characterization

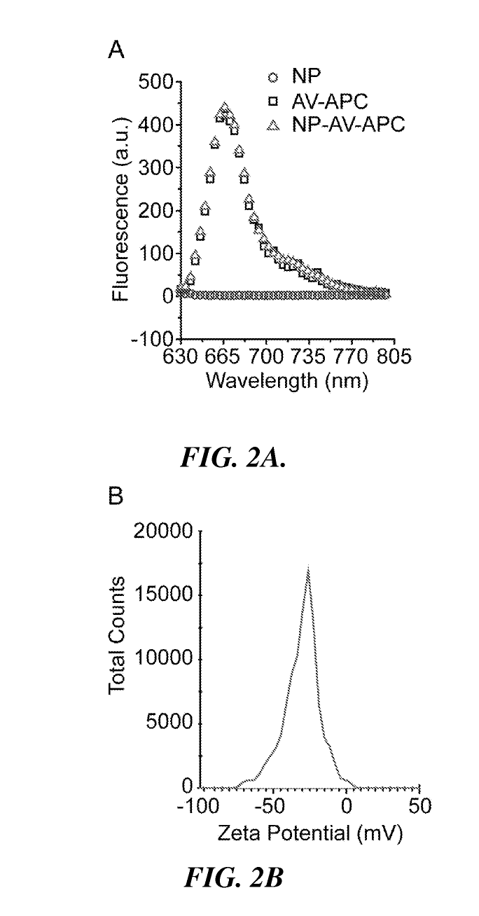

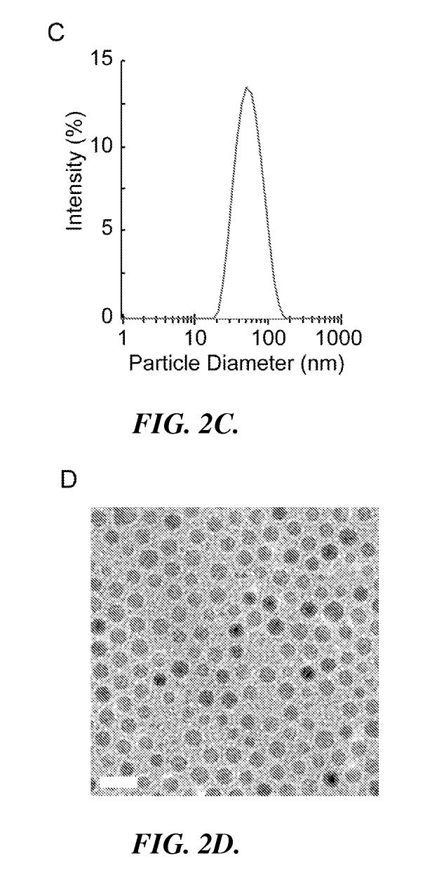

Physiochemical properties of NPs such as size and surface charge are important factors that determine the pharmacokinetics and cellular uptake of NPs. In a representative embodiment, fluorophore-labeled streptavidin (AV-APC) immobilized on the surface of iron oxide NPs (NP-AV-APC) was used to identify and bind to the biotinylated targeting moiety, GPC3 antibody. Streptavidin (AV), a tetrameric protein with an isoelectric point value of 5, is well known for its strong non-covalent binding to biotin with a dissociation constant of 4×10⁻¹⁴ M. The immobilization of streptavidin on the surface of the NP was confirmed by fluorescence measurements of NP, AV-APC and NP-AV-APC after purification (FIG. 2A). While naked NPs show no fluorescent emission, both AV-APC and NP-AV-APC have a strong emission peak centered at about 665 nm, confirming the presence of AV-APC on the surface of NP-AV-APC. The zeta potential and hydrodynamic size of NP-AV-APC was measured. The NP-AV-APC construct had a zeta potential of −30.4 mV±12.4 in DI water at pH 7.0 (FIG. 2B). This negative charge is a targeting advantage because decreased surface charge of NPs reduce non-specific uptake by cells (typically negatively charged) and increase deep tissue penetration by reduced electrostatic attraction to extracellular matrices. The mean hydrodynamic size measured in PBS was 50.4 nm with a polydispersity index (PDI) of 0.13 (FIG. 2C). NP diameters between 20 and 150 nm are preferred, as diameters less than 20 nm have been shown to be susceptible to renal clearance while NPs with diameters less than 150 nm can extravasate from vasculature into the tumor microenvironment via enhanced permeability and retention (EPR) effect and avoid clearance by the RES. The hydrodynamic size corresponds well with the NP core diameter (about 12.5 nm) determined by TEM (FIG. 2D). Differences in the hydrodynamic size and the core size measured by TEM are attributed to hydration of the polyethylene glycol (PEG) coating and the addition of streptavidin on the polymer layer. In addition to core diameter, TEM also demonstrates relative monodispersity of the NP and a lack of particle aggregation, due to both the anti-fouling PEG conjugation and the electrostatic repulsion between highly negatively charged NPs.

GPC3 Expression in Hep G2 and HLF Cells

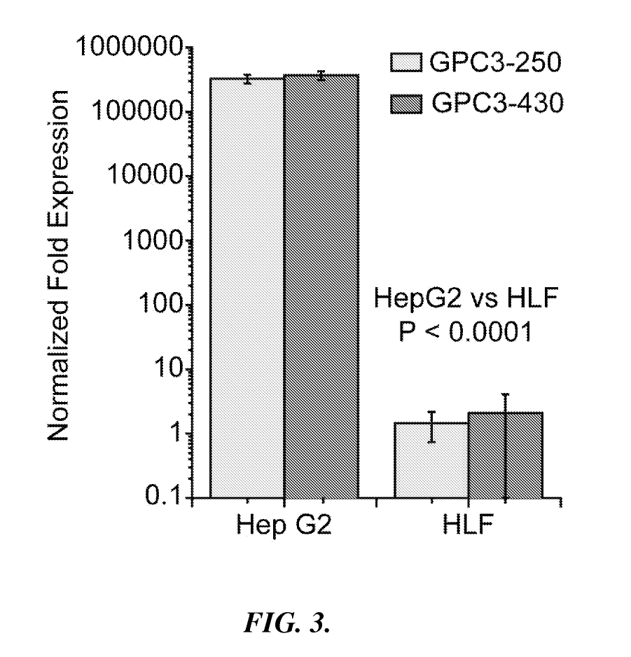

GPC3 was chosen as a molecular target for HCC based on the accessibility of its cell surface configuration to antibody-mediated targeting and its differential over-expression in most HCC cells compared to its absence in normal adult tissues. Immunohistochemical diagnosis of HCC using monoclonal GPC3 antibody staining has been effective in differentiating HCC from benign processes and other malignancies with 97% specificity. Microarray genomic profiling studies have confirmed considerable up-regulation of GPC3 expression in most human HCC specimens, implicating its role in pathogenesis and potential as an important biomarker and therapeutic target. The differential levels of GPC3 expression in Hep G2 (high) and HLF (negligible) cells has previously been reported, and consequently the Hep G2 and HLF cell lines have been used as GPC3-positive and GPC3-negative lines, respectively. Quantitative Real-time PCR was performed to confirm this finding in the cells described herein. Two distinct PCR primer sets for GPC3 resulting in two non-sequence-overlapping PCR products measuring 250 base-pairs and 430 base-pairs, respectively, were used to validate the results. FIG. 3 demonstrates the expression level of the GPC3 gene in both cell lines normalized to the expression level of the housekeeping gene GAPDH. GPC3 expression in Hep G2 cells was significantly elevated (320,000-fold increase) compared to that of the housekeeping gene GAPDH, whereas GPC3 expression in the HLF cells was equivalent to that of GAPDH. Both 250 and 430 base-pair PCR primer sets produced equivalent results (Hep G2 vs. HLF, P<0.0001), further validating these results.

Flow Cytometry Experiments

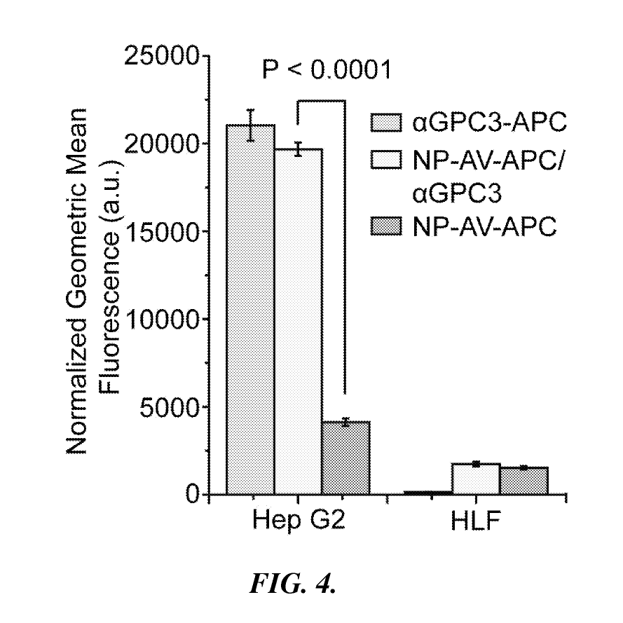

Specific targeting of the GPC3-positive Hep G2 cells using αGPC3 was demonstrated using flow cytometry. First, Hep G2 and HLF cells were treated with αGPC3-APC to establish feasibility of cell surface targeting using this antibody. As illustrated in FIG. 4, the level of fluorescence, expressed as the normalized geometric mean, is significantly higher in the GPC3-positive Hep G2 cells compared to the GPC3-negative HLF cells (P<0.0001), confirming the results of the PCR experiment. These findings suggest that the GPC3 expressed on the cell surface of Hep G2 cells can clearly be targeted and labeled by αGPC3 indicating its high affinity to GPC3. The GPC3 negative HLF cells are not labeled by αGPC3-APC, denoting this monoclonal antibody's specificity towards GPC3. In the group designated NP-AV-APC/αGPC3, the cell surface marker GPC3 was first labeled using the biotinylated αGPC3 in a pre-targeting step, then subsequently the biotin was targeted using the streptavidin iron oxide NP construct. This two-step pre-targeting approach sequentially employing biotinylated αGPC3 followed by streptavidin-conjugated NP's to specifically target GPC3-expressing HCC cells is illustrated in FIG. 1C. Pre-conjugated NP-AV-APC/biotin-αGPC3 particles showed negligible targeting effect for Hep G2 cells, compared to the two-step pre-targeting approach. This finding may be attributed to the NP-AV-APC/biotin-αGPC3 complexes being subject to significant electrostatic repulsion due to the highly negative charged NP-AV-APC and cell surface interactions, which in turn could limit the interaction of αGPC3 with GPC3 on the cell membrane. Additionally, steric hindrance between bulky groups such as NPs (MW 2.8×10⁶ based on its 12 nm core diameter) and streptavidin (MW 6×10⁴) near the antibody could also inhibit antibody-antigen interactions. However, biotin-streptavidin interactions are much stronger than that between antibody-antigen, which could overcome the interfering charge repulsion limiting the opportunity of interaction between NPs and the cell surface. As shown in FIG. 4, in Hep G2 cells, there was clear demonstration of GPC3-specific targeting by the NP as evidenced by a four-fold increase in the mean fluorescence in the targeted NP-AV-APC/αGPC3 group compared to the non-targeted NP-AV-APC control group (P<0.0001) using the pre-targeted approach. No difference in mean fluorescence was observed between the targeted NP-AV-APC/αGPC3 group and NP-AV-APC control group for the GPC3-negative HLF cells.

Confocal Fluorescence Imaging

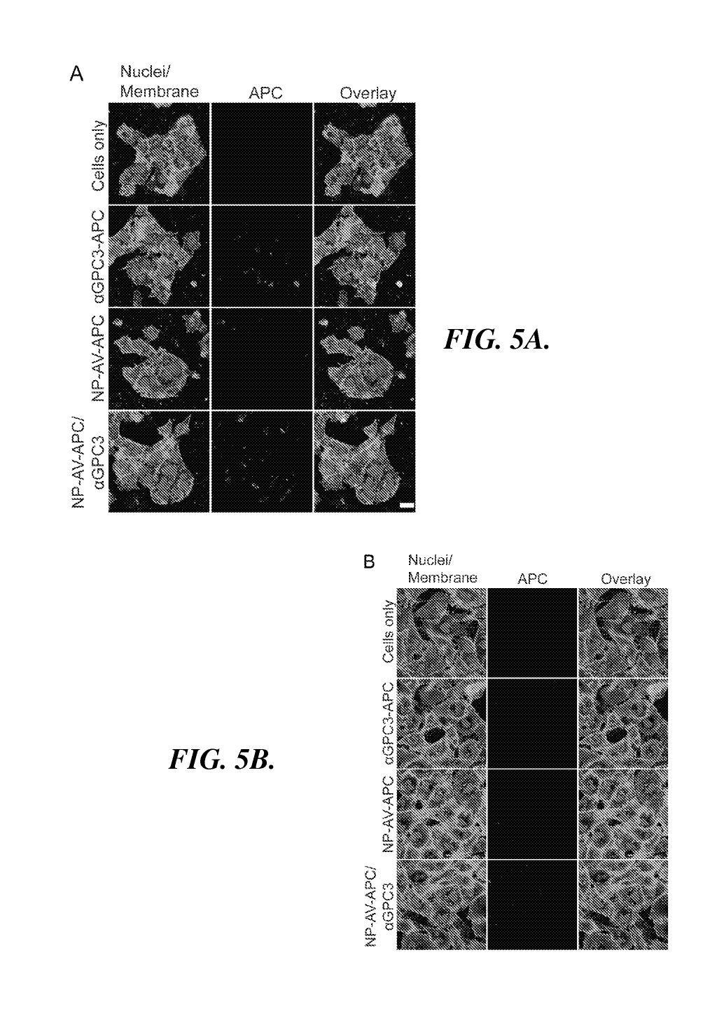

NIR fluorophore was immobilized onto NPs as a dual imaging agent, which could endow enhanced image resolution for clinical application. Targeted cellular labeling using αGPC3 and cellular localization of either αGPC3-APC or NP-AV-APC/αCPC3 was visualized by confocal fluorescence microscopy. Fluorescence images of Hep G2 (FIG. 5A) and HLF (FIG. 5B) cells further confirmed the results of the flow cytometry studies. The GPC3-positive Hep G2 cells treated with αGPC3-APC demonstrated cell membrane labeling, whereas the GPC3-negative HLF cells did not. Hep G2 cells treated with biotinylated αGPC3 followed by NP-AV-APC demonstrated cell membrane labeling with fluorophore. Hep G2 cells treated with the control NP-AV-APC demonstrated negligible fluorescence, indicating that the NP-AV-APC itself exhibited low non-specific uptake or binding by these cells. The HLF cells were not labeled by either the αGPC3-APC or the biotinylated αGPC3 bound by NP-AV-APC particles. In summary, the fluorophore labeling seen on imaging by either the APC-labeled αGPC3 or the biotinylated αGPC3 bound to NP-AV-APC was specific to the GPC3-expressing Hep G2 cells.

In Vitro MRI

To ensure that the NP-AV-APC construct retains sufficient magnetism after cell surface labeling, and to test the potential use of these particles as a GPC3-specific MRI contrast agent for HCC, the magnetic properties of the NP-AV-APC was studied. R₂ relaxation was evaluated at 4.7 T by acquiring phantom MR images of agarose with 1×10⁶ cells per each of the four treatment groups for both Hep G2 and HLF cells. The colorized R₂ maps in FIG. 6A and quantitative R2 calculations in FIG. 6B demonstrate significantly increased relaxivity measurements for Hep G2 cells treated with biotinylated αGPC3 followed by NP-AV-APC particles compared to other conditions and to HLF cells. For Hep G2 cells in the NP-AV-APC/αGPC3 group, there was a three-fold increase in R₂ values over untreated cells and a significant increase over cells treated with non-targeted NP-AV-APC. HLF cells in the NP-AV-APC/αGPC3 group demonstrated an increase in R₂ value over those in the cells only group. However, the uptake was not significantly different than the R₂ value in the non-targeted NP-AV-APC group. These results suggest that a greater number of magnetic NPs bound to the cell surface of Hep G2 cells in the NP-AV-APC/αGPC3 group compared to the non-targeted NP-AV-APC group. Additionally, a greater number of magnetic NPs bound to the Hep G2 cells in the NP-AV-APC/αGPC3 group compared to the HLF groups treated with NP-AV-APC/αGPC3 and NP-AV-APC, confirming GPC3-specific targeting and demonstrating the potential of this NP construct as an effective MRI contrast agent.

The present invention demonstrates that GPC3, a surface receptor over-expressed in most HCCs, can be employed as a molecular target to specifically identify HCC cells using a multifunctional NP. This NP construct has a number of advantages in its design and application. First, the target ligand GPC3 is a proteoglycan expressed on the cell surface of most HCCs, potentially making it widely applicable to most HCC patients. In contrast, normal adult human cells and tissues, and even dysplastic nodules found in cirrhotic livers that often account for indeterminate lesions encountered on conventional CT or MR imaging, lack GPC3 expression, making GPC3 a potentially powerful tool to discriminate between these benign entities and HCC. The present invention demonstrates the ability of the αGPC3 to target GPC3-expressing HCC cells with high specificity. Second, the characteristics of the NP construct are optimal for targeted delivery to the cells of interest, and an ideal target-specific imaging contrast agent. The hydrodynamic size of 50 nm is sufficiently large that it will not be quickly filtered out through the glomeruli, while sufficiently small that it can evade the RES clearance by the EPR effect. A negative zeta potential also minimizes unwanted non-specific binding and uptake by surrounding tissues, and allows for deeper tissue penetration to the target. The NP construct also has the unique advantage of dual modality imaging: targeted MR imaging which can be used for pretreatment staging/planning and targeted NIRF imaging which can be used in the operating theater to ensure adequate surgical margins during tumor resection.

The following examples are provided for the purpose of illustrating, not limiting, the invention.

### EXAMPLES

### Example 1

### Preparation, Characterization, and Use of Representative Nanoparticle and Labeled Targeting Agent for Imaging HCC Cells

In this example, the preparation and characterization of a representative nanoparticle (NP-AV-APC) and labeled targeting agent (biotinylated αGPC3) useful in the methods and systems of the invention is described. Also described is the use of the representative nanoparticle and labeled targeting agent in a method for imaging HCC cells in which HCC cells are pre-targeted with the labeled targeting agent and then labeled with nanoparticle having dual functionality (fluorescence and magnetic resonance imaging capabilities).

Preparation of Iron Oxide NPs and Surface Modification

Iron oxide NPs were prepared as described in Fang C., et al., “Functionalized nanoparticles with long-term stability in biological media,” Small, 2009 5(14):1637-41. Briefly, oleic acid-coated NPs synthesized by thermal decomposition of iron oleate complex were reacted with triethoxysilylpropyl succinic anhydride (SAS, Gelest, Morrisville, Pa.) using ligand-exchange and condensation to form hydrophilic SAS-coated NPs. Amine-functionalized polyethylene-glycol (PEG) was attached via N,N-dicyclohexylcarbodiimide (DCC)-mediated coupling. The NP concentration was adjusted to 4 mg/mL in 150 mM boric acid, pH 8.0. SIA (4 mg, Invitrogen) dissolved in 100 μL DMSO was added to 1 mL NP solution in an amber glass vial and reacted at room temperature (RT) overnight on a rotator. A Sephracyl-200 column (GE Healthcare, Piscataway, N.J.) was eluted with 25 mL 150 mM boric acid, pH 8.0, and the iodoacetate functionalized NPs were purified using the column.

Streptavidin (AV) (5 mg, lyophilized in 10 mM phosphate buffered saline (PBS), pH 7.4, Invitrogen) was dissolved in 1 mL deionized (DI) water. Alexa Fluor 647 (0.5 mg, Invitrogen) was dissolved in 100 μL DMSO and 50 μL was added to the AV. The reaction was covered in foil and placed on a shaker for 1 hr at RT. A PD-10 desalting column (GE Healthcare) was eluted with 25 mL 50 mM sodium bicarbonate, pH 8.5, and the fluorophore labeled AV was purified. 60 μL of N-succinimidyl-S-acetylthioacetate (SATA, Invitrogen) dissolved in DMSO at 1 mg/mL was added to the purified fluorophore (Alexa Fluor 647) labeled AV (AV-APC) and reacted on a shaker for 2 hrs at RT. To a 1 mg aliquot of purified AV-APC, 55 μL of deprotection solution (0.5 M hydroxylamine, 25 mM EDTA, pH 7.2) was added, reacted for 40 min, and then Zeba spin column purified. The NP-SIA concentration was adjusted to 2 mg/mL and then added to the purified AV-APC in an amber vial and reacted overnight on a shaker at RT. The AV-APC labeled NP (NP-AV-APC) was purified with a Sephracyl-200 column. The Fe concentration was determined by inductively coupled plasma atomic emission spectroscopy (ICP-AES, Thermo Jarrell Ash Corporation, Franklin, Mass.). FIG. 1A illustrates the preparation of NP-AV-APC.

NP Characterization and Transmission Electron Microscopy

The fluorescence spectra of NP, AV-APC and NP-AV-APC were measure with an excitation wavelength of 600 nm using the SpectraMax M2 microplate reader (Molecular Devices, Sunnyvale, Calif.). The surface charge and hydrodynamic size of the NPs was measured in PBS using a Zetasizer Nanoseries dynamic light scattering particle size analyzer (Malvern, Worcestershire, UK). The αGPC3-NP were loaded onto a grid and then dried in a dessicator at room temperature for transmission electron microscopy (TEM) analysis. TEM samples were observed on a Phillips CM100 TEM (Phillips, Eindhoven, The Netherlands) operating at 100 kV with a Gatan 689 digital slow scan camera.

Cell Culture

Human HCC cell lines, Hep G2 (ATCC, Manassas, Va.) and HLF (Japanese Collection of Research Bioresources, Shinjuku, Japan) were maintained in Eagle's minimum essential medium (MEM) and Dulbecco's modified essential medium (DMEM), respectively, supplemented with 10% (v/v) fetal bovine serum (FBS, Atlanta Biologicals, Lawrenceville, Ga.) and 100 units/ml penicillin/streptomycin (Invitrogen, Carlsbad, Calif.). The cell cultures were incubated at 37° C. atmosphere supplemented with 5% CO₂, and subcultivated at a ratio of 1:4 twice weekly using TrypLE (Invitrogen). Hep G2 cells were passed through a 25 G needle to minimize clumping. For experiments, the cells were detached using Versene-EDTA 1:5000 (Invitrogen) to minimize enzymatic cleavage of the GPC3 expressed on the cell surface.

Quantitative Real-Time PCR

Cells were detached using TrypLE (Invitrogen), and homogenized by passing through QIAshredder columns (Qiagen, Valencia, Calif.). Total RNA was isolated from Hep G2 and HLF cells in triplicate using RNeasy (Qiagen), and 30 ng of total RNA for each sample was converted to cDNA using the iScript cDNA Synthesis Kit (Bio Rad, Hercules, Calif.) following the manufacturer's instructions. iQ SYBR Green Supermix (Bio Rad) was used for template amplification with a primer for each of the transcripts examined. Thermocycling for all targets were carried out in a solution of 30 μl containing 0.3 μM primers (Integrated DNA Technologies, San Diego, Calif.) and 4 pg cDNA from the reverse transcription reaction under following conditions: 15 sec at 94° C., 30 sec at 55° C., and 30 sec at 72° C. The reaction was monitored in real-time using a CFX96 (BioRad). Two sets of GPC3 primers with gene product sizes of 250 and 430 base-pairs without any gene sequence overlap were used to confirm the presence of this gene. Primer nucleotide sequences were as follows: GPC3 250 sense 5′-GATACAGCCAAAAGGCAG-3′ (SEQ ID NO:1), antisense 5′-ATCATTCCATCACCAGAG-3′ (SEQ ID NO:2), GPC3 430 sense 5′-GCAAGTATGTCTCCCTAAGG-3′ (SEQ ID NO:3), antisense 5′-AGGTCACGTCTTGCTCCTC-3′ (SEQ ID NO:4). GPC3 expression was normalized to expression of the house keeping gene GAPDH sense 5′-TCGACAGTCAGCCGCATCTTCTTT-3′ (SEQ ID NO:5), antisense 5′-ACCAAATCCGTTGATCCGACCTT-3′ (SEQ ID NO:6).

Bioconjugation of GPC3 Antibody to Biotin

Purified mouse anti-human monoclonal IgG_{2A} antibody to GPC3 (αCPC3, 100 μg lyophilized, R&D Systems, Minneapolis, Minn.) was resuspended in 100 μL 0.1 M NaHCO₃ (Sigma-Aldrich, St. Louis, Mo.). 6-((6-((Biotinoyl)amino)hexanoyl)amino) hexanoic acid, succinimidyl ester, a biotin moiety with a 14-atom spacer consisting of two aminohexanoic acid chains (Biotin-XX, SE, MW: 567.70, 8.4 mg, Invitrogen) was dissolved in 200 μL DMSO (Sigma-Aldrich), and then diluted with addition of 800 μL DI H₂O. 1.85 μL of the biotin-XX-DMSO solution was added to the αGPC3 and incubated at RT for 15 min. A desalting spin column (Zeba 0.5 mL, 7K MW exclusion limit, ThermoScientific, Rockford, Ill.) was equilibrated with 300 μL 0.1 M sodium bicarbonate washes at 4000 rpm for 1 min, and then the biotinylated αGPC3 was passed through the column. 25 μL of the purified product was diluted to 1 mL with FACS buffer (MEM with 2% FBS and 0.04% human IgG (Invitrogen)) for cell labeling experiments. FIG. 1B illustrates the biotinylation of αGPC3.

Cell Labeling

Hep G2 and HLF cells were detached using Versene, resuspended in MEM with 10% (v/v) FBS, and counted with Trypan blue exclusion, confirming viability. Cells were centrifuged at 650 rcf for 7 min and the pellets were resuspended in FACS buffer at a cell concentration of 4×10⁶ cells/mL for flow cytometry, or 1×10⁷ cells/mL for MRI experiments, and incubated on ice for 15 min with periodic gentle mixing. For the NP-AV-APC/αGPC3 group, purified biotinylated αGPC3 was added to the cells in a pre-targeting step and incubated on ice for 60 min with periodic gentle mixing. Cells were then washed with FACs buffer and centrifuged at 650 rcf for 7 min. Pellets were resuspended in FACS buffer containing NP-AV-APC and incubated on ice for 30 min with periodic gentle mixing. For the αGPC3-APC group, 40 μL of allophycocyanin (APC) labeled αGPC3 (R&D Systems) was added to the cells and incubated on ice for 30 min with periodic gentle mixing. For the control NP-AV-APC group, untreated cell pellets were resuspended in FACS buffer containing NP-AV-APC and incubated on ice for 30 min with periodic gentle mixing. All cell group samples were washed twice with FACS buffer and centrifugation, then resuspended in FACS buffer and fixed with equal volume of 4% formaldehyde. All experiments were performed in triplicate. The pre-targeting scheme for the NP-AV-APC/αGPC3 group is illustrated in FIG. 1C.

Flow Cytometry

GPC3-targeted labeling of the cells was quantified using flow cytometry. Fixed cells (4×10⁵) were analyzed with a BD FACSCanto flow cytometer (Becton Dickinson Biosciences, Franklin Lakes, N.J.) equipped with a 633 nm red diode laser for excitation of Alexa Fluor 647 and APC fluorophores, appropriate band-pass filters to collect the emission wavelengths, and a digital instrument using FACSDiva software. A minimum of 30,000 cells was analyzed for each sample, and triplicate samples were evaluated for each treatment group. Data analysis was performed on FlowJo Version 8.8.6 (Tree Star, Ashland, Oreg.).

In Vitro Fluorescence Microscopy Imaging

Cells from the four treatment groups (Cells only, αGPC3-APC, NP-AV-APC/αCPC3, and NP-AV-APC) were plated on sterile 24 mm glass cover slides (5×10⁵ per slide) and grown for 24 hrs at 37° C. in humidified atmosphere with 5% CO₂. Cells were washed with PBS three times, and fixed in 4% formaldehyde/PBS solution (methanol free, Polysciences Inc., Warrington, Pa.) for 30 min. The fixative was then removed, and cells were washed again with PBS three times. Cell membranes were then labeled with Alexa Fluor 555 wheat germ agglutinin (Invitrogen) according to the manufacturer's instructions. The slides were then mounted with 4′,6-diamidino-2-phenylindole (DAPI)-containing Prolong Gold antifade solution (Invitrogen) for cellular nuclei staining and fluorescence preservation. The slides were examined by fluorescence microscopy using a Zeiss LSM 510 Meta confocal fluorescence microscope (Zeiss, Peabody, Mass.) equipped with 405 nm diode, 458 nm, 488 nm, 514 nm Argon/2, and 633 nm HeNe laser lines for excitation, and appropriate band-pass filters for collection of DAPI, Alexa Fluor 555, and NIRF (Alexa Fluor 647 and APC) emission signals. In all fluorescence images the DAPI signal is depicted in blue, Alexa Fluor 555 signal in green, and the NIRF signal in red.

In Vitro MRI

Cell pellets (10⁶ cells) were suspended in 50 μL of 1% agarose casts. T₂ relaxation measurements were performed on a 4.7-T Bruker magnet (Bruker Medical Systems, Karlsruhe, Germany) equipped with Varian Inova spectrometer (Varian, Inc., Palo Alto, Calif.). A 5-cm volume coil and spin-echo imaging sequence were used to acquire T₂-weight images. Images were acquired using a repetition time (TR) of 3000 ms and echo times (TE) of 14.0, 20.0, 40.0, 60.0, 100.0 and 150.0 ms. The spatial resolution parameters were: acquisition matrix of 256×128, field-of-view of 35×35 mm, section thickness of 1 mm and two averages. The T₂ map was generated by ImageJ software (National Institute of Health, Bethesda, Md.) based on the equation, SI=A·exp(−TE/T₂)+B, where SI is the signal intensity, TE is the echo time, A is the amplitude, and B is the offset. The R₂ map was generated by taking the reciprocal of the T₂ map.

### Example 2

### Preparation of a Representative Nanoparticle for Imaging HCC Cells

In this example, the preparation of a representative nanoparticle (chitosan-grafted PEG NP) useful in the methods and systems of the invention is described.

The iron oxide nanoparticles were synthesized and coated with the chitosan-PEG polymer by co-precipitating Fe(II) and Fe(III) with the addition of sodium hydroxide in the presence of the polymer, as described in U.S. patent application Ser. No. 12/384,923, filed Apr. 9, 2009 (US 2010/0260686), expressly incorporated herein by reference in its entirety. The chitosan-PEG coated nanoparticles (NPs) were then transferred into a 0.1M sodium bicarbonate pH 8.5 buffer by gel permeation chromatography (GPC) using Sephacryl S200 medium (GE Healthcare, Piscataway, N.J.).

The NPs can be fluorescently labeled with Alexa Fluor 647 (AF647) by dissolving 1 mg AF647 succinimidyl ester (Invitrogen, Carlsbad, Calif.) in 0.15 mL dimethylsulfoxide (DMSO) and mixing with the NPs (3.5 mg Fe; about 2 mg Fe/mL) at room temperature for 2 hrs. Free AF647 can be removed by GPC purification using phosphate buffered saline (PBS) pH 7.4 (Sephacryl S200).

### Example 3

### Method for Preparing a Representative Anti-Glypican-3 Antibody: GPC3 IgG₁

In this example, methods for preparing a representative anti-glypican-3 antibody (GPC3 IgG₁) and related functional fragments are described.

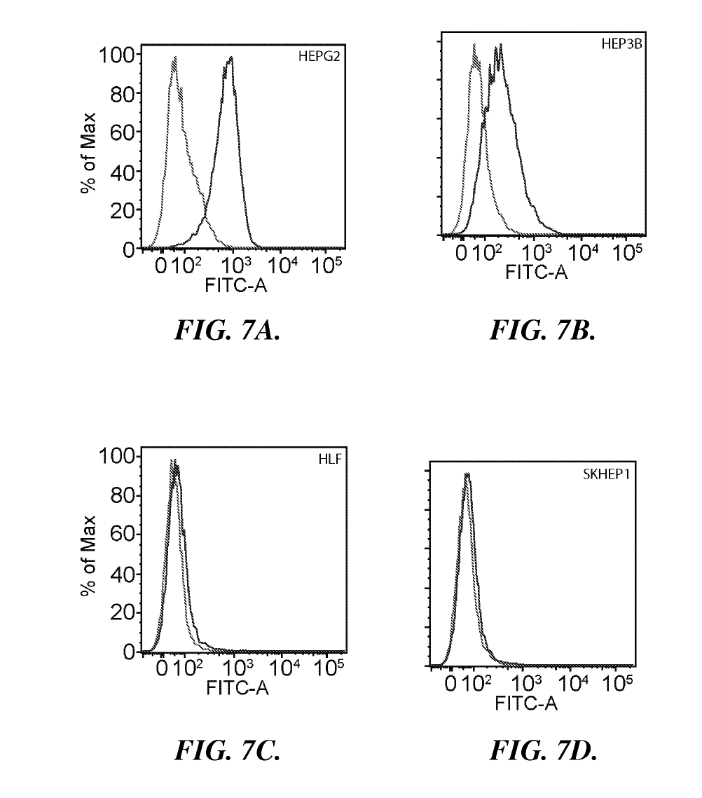

GPC3 IgG₁. GPC3 IgG₁ producing hybridomas were generated using conventional methods. Briefly, RBF/DnJ mice were immunized with recombinant human GPC3 protein in Freund's adjuvant. Following several boost injections, antiserum ELISAs confirmed presence of the GPC3 IgG. Additional boosts were delivered to ensure IgM/IgG switch, verified on ELISA with IgG titrated 1:10,000. Following final pre-fusion boosts, mice were euthanized, spleens were harvested, and 10⁸ splenocytes were fused 1:1 with FOX-NY myeloma cells, and resultant hybridomas were resuspended in adenine/aminopterin/thymidine FBS. Clones producing high GPC3 IgG₁ titers were selected using capture ELISA with goat anti-mouse IgG₁ for isotyping. FACS repeated with protein A purified GPC3 IgG₁ from these hybridoma clones demonstrates specific binding to GPC3 expressing cells (FIGS. 7A-7D). Mouse IgG₁-FITC was used as an isotype control for these experiments, performed in triplicate.

Fab and F(ab′)₂ Production. Given that mouse IgG₁ is resistant to Pepsin (absent cleavage site Leu₂₃₄), the antigen binding fragments of GPC3 were generated using Ficin, a sulfhydryl protease that produces F(ab′)₂ vs. Fab at 4 vs. 25 mM cysteine. GPC3 IgG₁ applied to resin immobilized Ficin columns equilibrated in cysteine HCl buffer was incubated at 37° C. 5 hrs for Fab, 30 hrs for F(ab′)₂. Digestion products were purified with protein A columns to remove Fc and undigested IgG₁. 30 kDa ultra centrifugal filters were used to concentrate the fragments. Fab and F(ab′)₂ with the predicted molecular weights were confirmed on SDS-PAGE.

### Example 4

### Conjugation of Anti-Glypican-3 Antibody to Magnetic Nanoparticles

In this example, a method for conjugating a representative anti-glypican-3 antibody (GPC3 IgG_{2A}) to a representative magnetic nanoparticle, prepared as described above in Example 1, is described.

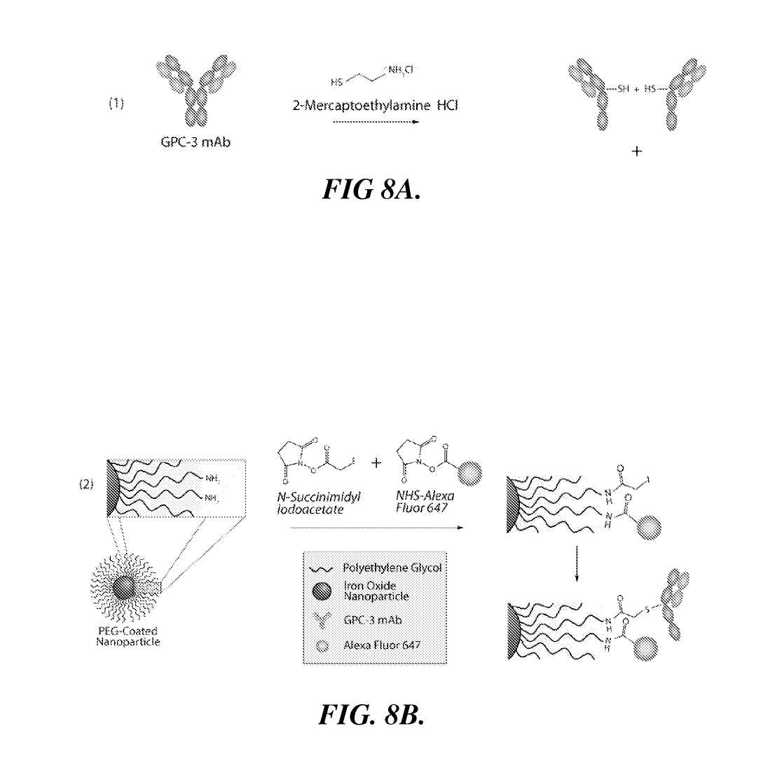

2-Mercaptoethylamine-HCl (MEA) treated Fc intact GPC3 IgG_{2A} was conjugated to PEGylated iron oxide NPs. MEA cleaves the GPC3 IgG at the hinge disulfide bond, leaving a sulfhydryl group for use in conjugation (see FIG. 8A). SIA was used to conjugate the IgG element to the functionalized amine groups on the PEG coating of NPs and the reactive NHS ester of near-infrared fluorophore Alexa Fluor 647 (AF647) was used to conjugate the fluorophore to the nanoparticle (see FIG. 8B).

While illustrative embodiments have been illustrated and described, it will be appreciated that various changes can be made therein without departing from the spirit and scope of the invention.

The embodiments of the invention in which an exclusive property or privilege is claimed are defined as follows:

1. A method for detecting liver cancer cells, comprising:
(a) contacting liver cancer cells with a magnetic nanoparticle to provide liver cancer cells labeled with the nanoparticle, wherein the nanoparticle is associated with an anti-glycipan-3 antibody or functional fragment thereof bound to the liver cancer cells; and
(b) magnetic resonance imaging of the labeled liver cancer cells to detect the liver cancer cells, wherein the method has the capacity to detect about 1×10⁶ liver cancer cells.

2. The method of claim 1, wherein contacting the liver cancer cell with magnetic nanoparticle comprises administering the magnetic nanoparticle to a subject to be diagnosed or treated.

3. The method of claim 2, wherein administering the magnetic nanoparticle comprises intravenous administration.

4. The method of claim 1, wherein contacting the liver cancer cell with magnetic nanoparticle comprises administering the magnetic nanoparticle associated with an anti-glycipan-3 antibody or functional fragment thereof to a subject to be diagnosed or treated.

5. The method of claim 4, wherein administering the magnetic nanoparticle associated with an anti-glycipan-3 antibody or functional fragment thereof comprises intravenous administration.

6. The method of claim 1, wherein the anti-glycipan-3 antibody is an IgG₁.

7. The method of claim 1, wherein the functional fragment of the anti-glycipan-3 antibody is selected from the group consisting of Fab and F(ab′)₂ fragments.

8. The method of claim 1, wherein the anti-glycipan-3 antibody is covalently coupled to the nanoparticle.

9. The method of claim 1, wherein the anti-glycipan-3 antibody is associated to the nanoparticle through an affinity binding interaction.

10. The method of claim 1, wherein the magnetic nanoparticle comprises a material is selected from the group consisting of ferrous oxide, ferric oxide, silicon oxide, polycrystalline silicon oxide, silicon nitride, aluminum oxide, germanium oxide, zinc selenide, tin dioxide, titanium, titanium dioxide, nickel titanium, indium tin oxide, gadolinium oxide, stainless steel, gold, and mixtures thereof.

11. The method of claim 1, wherein the nanoparticle further comprises an imaging agent.

12. The method of claim 11, wherein the imaging agent is select from the group consisting of a magnetic resonance imaging agent, a fluorescence imaging agent, an ultrasound imaging agent, a radiolabel, a surface plasmon resonance imaging agent, and combinations thereof.

13. The method of claim 1, wherein the nanoparticle comprises a core having a surface and a coating on the surface of the core, the coating comprising a copolymer comprising a chitosan and a poly(ethylene oxide) oligomer.

14. The method of claim 13, wherein the copolymer is a graft copolymer having a chitosan backbone and poly(ethylene oxide) oligomer side chains.

15. A method for labeling liver cancer cells, comprising:
(a) contacting liver cancer cells with an anti-glycipan-3 antibody or functional fragment thereof to provide liver cancer cells labeled with the anti-glycipan-3 antibody or functional fragment thereof, wherein the anti-glycipan-3 antibody or functional fragment thereof comprises a first binding partner;
(b) contacting the labeled liver cancer cells with a magnetic nanoparticle comprising a second binding partner to provide a nanoparticle-labeled liver cancer cells, wherein the first binding partner has a binding affinity toward the second binding partner effective to bind the nanoparticle to the liver cancer cells; and
(c) magnetic resonance imaging of the labeled liver cancer cells to detect the liver cancer cells, wherein the method has the capacity to detect about 1×10⁶ liver cancer cells.

16. The method of claim 15, wherein contacting the liver cancer cell with the anti-glycipan-3 antibody or functional fragment thereof comprises administering the anti-glycipan-3 antibody or functional fragment thereof to a subject to be diagnosed or treated.

17. The method of claim 16, wherein administering the anti-glycipan-3 antibody or functional fragment thereof comprises intravenous administration.

18. The method of claim 15, wherein contacting the labeled liver cancer cells with a magnetic nanoparticle comprises administering the magnetic nanoparticle to a subject to be diagnosed or treated.

19. The method of claim 18, wherein administering the magnetic nanoparticle comprises intravenous administration.

20. The method of claim 15, wherein the first binding partner is biotin or analog or derivative thereof, and wherein the second binding partner comprises an avidin, a streptavidin, a neutravidin, or functional fragment thereof.
